(19) United States
(12) Patent Application Publication
(10) Pub. No.: US 2025/0236899 A1
(43) Pub. Date: Jul. 24, 2025

(54) OBTAINING BACTERIAL CELLULOSE WITH KOMAGATAEIBACTER SP. GUS3

(71) Applicant: ISTANBUL SABAHATTIN ZAIM ÜNIVERSITESI, Istanbul (TR)

(72) Inventors: Eda BÜYÜKDUMAN, Istanbul (TR); Banu METIN, Istanbul (TR)

(73) Assignee: ISTANBUL SABAHATTIN ZAIM ÜNIVERSITESI, Istanbul (TR)

(21) Appl. No.: 18/848,160

(22) PCT Filed: Nov. 16, 2022
(86) PCT No.: PCT/TR2022/051306

§ 371 Date: Sep. 18, 2024

(30) Foreign Application Priority Data
Mar. 18, 2022 (TR) 2022/004180

(51) Int. Cl. C12P 19/04; C12N 1/20; C12R 1/02

(52) U.S. Cl. CPC C12P 19/04; C12N 1/205; C12R 2001/02

(57) ABSTRACT

A method for obtaining bacterial cellulose from the vinegar isolate Komagataeibacter sp GUS3, which produces 1.644±0,4016 g/L cellulose.

### TECHNICAL FIELD

This invention relates to obtaining the bacterial cellulose which is produced as an alternative to plant-derived cellulose as well as which is a biopolymer used and having the potential for use in the fields of food, medicine, pharmaceuticals, biotechnology, biomedical, paper, electronics, cosmetics and textiles by vinegar isolate Komagataeibacter sp. GUS3.

### BACKGROUND

Plant cellulose is a process obtained from wood with a large amount of efficiency, by applying a multi-step method that requires energy and cost, and also causes the generation of wastes that cause environmental pollution (Saied et al., 2004).

Bacterial cellulose differs from plant cellulose with its properties such as being purer, having a higher water retention capacity, being suitable for changes occurring during production, and being accepted as GRAS (Akoğlu et al., 2010). Cellulose fibrils produced by microorganisms are chemically similar to cellulose produced from wood pulp, but are different in many ways. The main differences include the cross-sectional width of these fibrils. It has an unusually large capacity to absorb aqueous solutions, with greater surface area than conventional wood pulp cellulose and with the natural hydrophilicity of cellulose. It has been stated that this high absorbent capacity is useful in the manufacture of dressings that can be used in the treatment of burns or as surgical dressings to prevent surface drying of organs during long surgical procedures.

Bacterial cellulose (BC) is a natural polymer produced by some microorganisms, which is biocompatible, easy to manufacture, has high tensile strength, and has high water retention with its nanofibril network structure. One of the most important factors affecting its production is the carbon and nitrogen sources used.

The production and use of biological preparations for residue-free production are becoming more widespread day by day. It is a is a biopolymer which is produced as an alternative to plant-derived cellulose, which has potential applications in the fields such as food, medicine, pharmaceuticals, biotechnology, biomedical, paper, electronics, cosmetics and textiles with its superior properties such as being economical, having elastic structure, surface area, water retention capacity, tensile strength, smaller pores and being obtained in a short time (Güzel and Akpinar, 2018; Bielecki et al. 2000).

BC can be used in many industrial applications, such as maintaining the particle size of food, cosmetics or coatings, fortifying foodstuffs, retaining moisture, improving food stability and developing food stability. It has a wide and reliable use potential in the food industry, especially in the production of low-calorie desserts, chips and confectionery, in the composition of desserts, ice cream and salad dressings as a filling, as well as in the coating of sausages and meats (Akoğlu et al., 2010). In medical fields, BC has the potential to be used as wound dressing, surgical wipes, treatment pad, burn bandage, tissue/organ cover and the like. In addition to being a useful wound dressing in the treatment of burns and injuries, BC can be applied as a sterile layer that can cool the skin surface, can keep the wound moist and protect it against microbial and virus loads that may come from outside, and BS is used in the production of fiber or yarn in the textile industry, in the production of surgical thread, in the cosmetics industry, as well as it can be used to make first quality paper or napkins (Güzel and Akpinar, 2018; Bielecki et al. 2000; Wen et al., 2015).

Cellulose fibrils produced by microorganisms have a very large capacity to absorb aqueous solutions with its small cross-section size, greater surface area than conventional wood pulp cellulose, and the natural hydrophilicity of cellulose. It is well known that the degree of polymerization of BC is higher than that of wood pulp and cotton linter used as industrial materials. Cellulose-containing polymeric materials with higher degrees of polymerization will generally have more excellent mechanical properties such as strength and elasticity. Cellulose synthesized by bacteria shows two times higher strength than plant-derived cellulose (Klemm et al., 2011). Therefore, BC is considered to be the strongest naturally synthesized biological material (Lee et al., 2014).

Currently, wood and cotton, which are industrially the main sources of cellulose, are processed and turned into products (Brown, 2004). In the method widely used in the industry, separation processes of lignin and hemicellulose from cellulose are performed (Saxena and Brown, 1997). Various pollutants are thrown into the air, soil and water during the pulp preparation processes applied to obtain cellulose from wood for industrial purposes (Keshk et al., 2006). Industrial wastes released during the production of paper and plant cellulose cause environmental pollution. Although bacteria can be easily grown on cheap waste materials and approximately 10 tons of bacterial polymers are obtained from a stagnant culture with a surface area of one hectare, growing only 600 kg of cotton in the same area and duration is important in terms of examining the economic dimension (Kudlicka, 1986). In this sense, cellulose produced by microorganisms is presented as an alternative source to plant cellulose (Saied et al., 2004). Intensive studies carried out on BC have shown that BC is chemically the same as plant cellulose, but differs in terms of its macromolecular structure and properties (Kudlicka, 1986). Today, cellulose is mainly obtained from plant sources. However, the fact that bacterial cellulose is more pure than plant cellulose, has high water retention capacity, is suitable for changes during production and is accepted as GRAS bring bacterial cellulose forefront (Akoğlu et al., 2010).

The application numbered EP3121265A1 relates to the microbiology and biotechnology industry. B17 strain isolated from Kombucha tea cell culture, identified as Komagataeibacter rhaeticus and stored in Latvia Microbial Strain Collection with number P 1463, is proposed as a promising bacterial cellulose (BC) producer. The cellulose synthesis capacity by strain P 1463 is quite high compared to another strain of Komagataeibacter hansenii B22.

In the application numbered U.S. Pat. No. 6,071,727, the process uses a rotating disk or linear carrier bioreactor containing a biological medium and a cellulose-producing microorganism to make a microbial cellulose with high water content and microbial cellulose.

### SUMMARY

Considering the applications made in the literature, new researches regarding the bacterial cellulose production of Komagataeibacter sp. GUS3 are required in terms of the development of usage areas.

The invention relates to bacterial cellulose production of the vinegar isolate Komagataeibacter sp. GUS3. Bacterial cellulose (BC) is a biopolymer which is produced as an alternative to plant-derived cellulose and which has potential for use (Güzel and Akpinar, 2018; Bielecki et al. 2000). GUS3 isolate (Komagataeibacter sp.) produces 1,644 (±0,402) g/L cellulose after one week of incubation under static conditions at 30° C. in 50 mL HS medium (Hestrin and Schramm, 1957) (pH 6).

### DETAILED DESCRIPTION OF THE EMBODIMENTS

Komagataeibacter sp. GUS3, which is the subject of the invention, is an isolate obtained from white grape vinegar.

Studies for the isolation of Komagataeibacter sp. GUS3, which is the subject of the invention: For the isolation of acetic acid bacteria Komagataeibacter sp. GUS3 from vinegar samples, 5 mL of homemade white grape vinegar was taken and homogenized with 45 mL of peptone water (0.1% peptone (Merck)) with a stomacher (Interscience). Afterwards, various dilutions prepared from the homogenate with peptone water were taken in amounts of 100 μL and added to AAM medium (acetic acid medium, 10 g/L glucose, 15 g/L peptone, 8 g/L yeast extract and 15 g/L agar, 0.3% acetic acid and 0.5% ethanol (Nielsen et al., 2007). Petris were then incubated at 30° C. for 2-4 days (Sharafi et al., 2010). A colony equal to the square root of the number of morphologically different colonies was selected from the petris that developed between 25 and 250 colonies, and three lines were added in other petris for purification. At the end of this period, it was selected from single colonies growing in petris and developed for 2-4 days at 30° C. in cryotubes containing 500 μL of AAM medium, and it was vortexed by adding glycerol so that the final volume contains 20% glycerol and stocked at −80° C.

Studies for molecular identification of the isolate which is the subject of the invention: Genomic DNA of the isolate was isolated using the PureLink™ Genomic DNA Mini Kit (Thermo Scientific). The 16S rRNA and 16S-23S rRNA ITS regions of the isolate were amplified by PCR (polymerase chain reaction). For 16S rRNA PCR, the primers 27F (5′-AGA GTT TGA TCC TGG CTC AG-3′) and 1492R (5′-GGT TAC CTT GTT ACG ACT T-3′) were used, and for 16S-23S ITS PCR, the primers 16S its1 (5′-ACC TGC GGC TGG ATC ACC TCC-3′) and 23S its2 (5′-CCG AAT GCC CTT ATC GCG CTC-3′) were used (Lane, 1991; Ruiz et al., 2000). PCR was prepared with 1× buffer, 0.2 mM deoxynucleoside triphosphate mix (dNTP), 2 μL each forward and reverse primer, ˜50 ng DNA, 2.5 U Taq DNA polymerase (Thermo Fisher Scientific) and 50 μL water to a final volume. PCR reactions were purified using the GeneJet PCR purification kit (Thermo Scientific) and sequenced by Sanger sequencing. Sequences were registered in the GenBank database with registration numbers of MZ396870 (16S rRNA) and MZ401140 (16S-23S rRNA ITS).

Studies while obtaining bacterial cellulose for the invention: In order to determine the temperature and ethanol resistance of the isolate, it was first developed in 50 mL enrichment medium (1% glucose, 1% yeast extract, and 1% ethanol) at 30° C. at 180 rpm for 72 hours. To determine the temperature stability, the culture grown was added in various dilutions of 4% (v/v) ethanol containing glucose, yeast extract (yeast extract), casein (GYEC) agar (1% glucose, 1% yeast extract, 2% calcium carbonate, and 2% agar, w/v) and it was left for incubation for 72 hours at 30° C., 35° C., and 38° C. Petris containing colonies that reproduce by forming a transparent zone around them are considered resistant to the specified condition. It was determined that Komagataeibacter sp. GUS3 can grow at 30° C. and 35° C., but it is not resistant to 38° C. For ethanol resistance, the isolate was added to GYEC agar containing 4, 6, 8, 10% and 12%, v/v ethanol, and it was left for incubation at 30° C. for 72 hours.

Komagataeibacter sp. GUS3 was determined to be resistant to ethanol up to 8%.

The invention includes the following process steps of obtaining bacterial cellulose with KOMAGATAEIBACTER SP.GUS3:
Use of medium (2% glucose, 0.5% peptone, 0.5% yeast extract, 0.34% Na₂HPO₄.2H₂O, and 0.115% citric acid monohydrate; w/v; pH 6.0) for cellulose production of the isolate,
the fact that the isolate is firstly grown in 5 mL HS medium at 30° C. for 2 days under static conditions for this,
and that it was then left for incubation at 30° C. for 7 days under static conditions by being transferred into 250 mL of erlenmeyer flasks containing 45 mL of HS medium,
that after the obtained cellulose biofilm is separated from the medium, it is washed with 2% NaOH at 80° C. for 45 minutes and washed with distilled water until pH reaches 7.
that the biofilm is then dried at 45° C. until it reaches a constant weight.

The isolate produces cellulose by 1.644±0.4016 Komagataeibacter sp. GUS3 at 28° C. Two separate experiments were carried out to calculate the mean and standard deviation of the isolate in the production of cellulose.

It is clear that a person skilled in the art can present the innovation revealed in the invention by using similar embodiments and/or can apply this embodiment to other fields with similar purposes used in the related art. Therefore, it is obvious that such embodiment will lack the criteria of innovation and especially overcoming the state of the art.

1-4. (canceled)

5. A method for obtaining bacterial cellulose with KOMAGATAEIBACTER SP. GUS3, with the method comprising the following process steps:
use of medium (2% glucose, 0.5% peptone, 0.5% yeast extract, 0.34% Na2HPO4.2H2O, and 0.115% citric acid monohydrate; w/v; pH 6.0) for cellulose production of the isolate,
grow the isolate is in 5 mL HS medium at 30° C. for 2 days under static conditions,
leave the isolate for incubation at 30° C. for 7 days under static conditions by being transferred into 250 mL of erlenmeyer flasks containing 45 mL of HS medium,
obtain a cellulose biofilm that is separated from the medium, is washed with 2% NaOH at 80° C. for 45 minutes, and washed with distilled water until pH reaches 7, and
dry the biofilm at 45° C. until it reaches a constant weight.

6. The method for obtaining bacterial cellulose with KOMAGATAEIBACTER SP. GUS3 according to claim 5, wherein the GUS3 isolate (Komagataeibacter sp.) contains 1,644 (±0,402) g/L cellulose after one week of incubation under static conditions at 30° C. in 50 mL HS medium pH 6.

7. The method for obtaining bacterial cellulose with KOMAGATAEIBACTE RSP. GUS3 according to claim 5, wherein, for the isolation of acetic acid bacteria Komagataeibacter sp. GUS3 from vinegar samples, 5 mL of homemade white grape vinegar is homogenized with 45 mL of peptone water (0.1% peptone (Merck)) with a stomacher; afterwards, various dilutions prepared from the homogenate with peptone water are taken in amounts of 100 μL and added to AAM medium; petris are then incubated at 30° C. for 2-4 days; and a colony equal to the square root of the number of morphologically different colonies is selected from the petris that developed between 25 and 250 colonies, and three lines are added in other petris for purification.

8. The method for obtaining bacterial cellulose with KOMAGATAEIBACTER SP. GUS3 according to claim 5, wherein the sequencing method of 16S rRNA and 16S-23S rRNA of the microorganism with the SANGER method is used.
